(19) United States
(12) Patent Application Publication
(10) Pub. No.: US 2013/0209581 A1
(43) Pub. Date: Aug. 15, 2013

(54) IODINATED ACTIVATED CHARCOAL FOR TREATING SYMPTOMS OF DEPRESSION

(71) Applicant: Staffan Skogvall, Lund (SE)

(72) Inventor: Staffan Skogvall, Lund (SE)

(73) Assignee: PHARMALUNDENSIS AB, Lund (SE)

(21) Appl. No.: 13/805,305

(22) PCT Filed: Jun. 21, 2011
(86) PCT No.: PCT/EP2011/060328

§ 371 Date: Feb. 19, 2013

(30) Foreign Application Priority Data
Jun. 24, 2010 (SE) 1050699-6

(51) Int. Cl. A61K 33/18; A61K 33/00; A61K 33/06

(52) U.S. Cl. CPC A61K 33/18; A61K 33/06; A61K 33/00
USPC 424/667

(57) ABSTRACT

The present invention relates to the use of iodinated activated charcoal, optionally in combination with a bromide salt, in treatment of one or more symptom(s) caused by depression, wherein said symptom(s) is/are selected from the group consisting depressive mood, emotional fatigue, mental fatigue which can be chronic, burnout, withdrawal from social situations, anxiety, concentration problems, unpleasant heart palpitations, irregular heart beats, a feeling of pressure in the chest, irritable bowel syndrome and sexual dysfunction.

### FIELD OF THE INVENTION

The present invention relates to the use of iodinated activated charcoal In treatment of symptoms of depression.

### BACKGROUND OF THE INVENTION

Clinical depression is a severe disorder characterized by an all-encompassing low mood, accompanied by low self-esteem, and by loss of Interest or pleasure in normally enjoyable activities (Depression, National Institute of Mental Health, U.S. Department of Health & Human Services National Institutes of Health NIH publication No. 08 3561 revised 2008). Other symptoms of depression include poor concentration and withdrawal from social situations and activities, reduced sex drive, digestive problems, headaches, fatigue and loss of energy (which sometimes is called burnout). In most countries the number of people who would suffer from depression during their lives falls within an 8-12% range (Andrade L, Caraveo-Anduaga J J, Berglund P. The epidemiology of major depressive episodes: Results from the International Consortium of Psychiatric Epidemiology (ICPE) Surveys. Int J Methods Psychiatr Res, 2003; 12(1):3-21).

The most popular idea about the cause of depression is a malfunction of the systems containing the transmitters serotonin, norepinephrine and dopamine in the brain (Nutt D J, Relationship of neurotransmitters to the symptoms of major depressive disorder. Journal of Clinical Psychiatry, 2008; 69 Supp E1:4-7). The cause to this malfunction is, however, unclear.

Depression is today mainly treated with anti-depressants, psychotherapy and electroconvulsive therapy. However, the treatment must continue for a long time, and a significant number of patients fail to recover completely, or experience new depressive episodes at a later point, The terms “refractory depression” and “treatment-resistant depression” are used to describe cases that do not respond to adequate courses of at least two antidepressants. In many major studies, only about 35% of patients respond well to medical treatment. (Wijeratne, Chanaka, Sachdev, Perminder. Treatment-resistant depression: Critique of current approaches. Australian and New Zealand Journal of Psychiatry. 2008;42(9):751-62). Thus, there is an urgent need for new and effective treatments.

U.S. Pat. Nos. 5,910,318 and 5,955,101 disclose starch-iodine pharmaceutical formulations for the preparation of capsules and tablets. The pharmaceutical formulations are suitable for administration to patients suffering from iodine deficiency diseases, in particular breast dysplasia, breast cancer, endometriosis, premenstrual syndrome, ovarian cysts and radiation sickness. In the formulation iodine is complexed with starch containing amylose, forming triiodide ions or polyiodide ions (I₅′ up to I₁₁′). Iodine is released in the upper small bowel after hydrolysis of the starch by α-amylase. Since the triiodide ions cannot exist in non-complexed form I₂ is released.

Iodine toxicity may be a concern when high amounts of iodine are administered to a human. Iodine toxicity is manifested by, among other symptoms, thyroiditis, goiter, hypothyroidism and hyperthyroidism. It has been suggested that some individuals can tolerate very high levels of iodine with no apparent side effects and that iodine intakes less than or equal to 1,000 mg/day are probably safe for the majority of the population, but may cause adverse effects in some individuals (Pennington, J. A., A Review of Iodine Toxicity Reports. Journal of the American Dietetic Association 1990 90(11):1571-81). Administration of a bromide salt, in particular sodium bromide, to animals suffering from the effects of iodine toxicity helps to reverse the symptoms (Baker, D. H., Parr, T. H. and Augspurger, N. R. Oral Iodine Toxicity in Chicks Can Be Reversed by Supplemental Bromine. Journal of Nutrition 2003 133:2309-2312). Sodium bromide is well tolerated by humans and it has been found to have a no-effect level of 4 mg/kg body weight (van Geideren, C. E., Savelkoul, T. J., Blom, J. L, van Dokkum, W. and Kroes, R. The No-effect Level of Sodium Bromide in Healthy Volunteers. Human & Experimental Toxicology 1993 12(1):9-14).

Activated charcoal is used in medical applications to treat poisoning and oral overdose of various medicaments. Activated charcoal has a very large surface area; 1 gram has a surface area of 300-2000 m² (Greenwood, N. N. and Earnshaw, A. Chemistry of the Elements, page 301, Pergamon Press, 1984).

Impregnated activated charcoals are carbonaceous adsorbents which have chemicals finely distributed on their internal surface (Henning and Schaefer Gas Separation & Purification 1993 Vol 7 No 4 235-240). The impregnation optimizes the existing properties of the activated charcoal giving a synergism between the chemicals and the charcoal.

Iodinated activated charcoal has been used for many years to bind mercury in gas.

Iodinated activated charcoal has recently been found to be efficient in producing bronchorelaxation in humans or animals affected by airway obstruction, such as chronic obstructive pulmonary disease or asthma (WO 2009/067067 and WO/2009/078782).

### SUMMARY OF THE INVENTION

In the work leading to the present invention it was found that iodinated activated charcoal, which also may be denoted as elemental iodine, I₂, on activated charcoal, administered to the intestine of a mammal suffering from depression, results in the mammal experiencing a distinct improvement of the depression and symptoms associated with it, such as improved mood, more energetic, improved stamina and less anxiety.

The present invention thus relates to iodinated activated charcoal for use in treatment of a symptom caused by depression.

The present invention also relates to the use of iodinated activated charcoal for the manufacture of a medicament for treatment of a symptom caused by depression.

The present invention also relates to iodinated activated charcoal manufactured for use in treatment of a symptom caused by depression.

The present invention also relates to a method for the treatment of a symptom caused by depression in a mammal, comprising administration of a pharmacologically effective amount of iodinated activated charcoal to the intestine of said mammal.

The symptom mentioned above may be one or several of the symptoms selected from the group consisting of depressive mood, emotional fatigue, mental fatigue, which can be chronic, withdrawal from social situations, burnout, anxiety, concentration problems, unpleasant heart palpitations, irregular heart beats, a feeling of pressure in the chest, irritable bowel syndrome and sexual dysfunction.

### DETAILED DESCRIPTION OF THE INVENTION

In this specification the term iodine refers to elemental iodine, I₂, and the term iodinated activated charcoal refers elemental iodine on activated charcoal. The iodine is impregnated or adsorbed into the activated charcoal. Throughout this application both with regards to the description and the claims activated carbon comprising adsorbed iodine is considered equivalent to iodinated activated charcoal.

According to the invention it is possible to treat a mammal in need of the treatment in question. According to some embodiments, which may be preferred, the mammal is a human.

As mentioned above, the invention relates to treatment of depression or treatment of a symptom caused by depression.

The expression “a symptom” used herein denotes at least one symptom. Thus, according to the invention it is possible to treat one, two or several symptoms caused by depression.

One symptom of depression that may be treated according to the invention is fatigue, and in particular emotional fatigue or mental fatigue, or a combination of both of these. The mental fatigue may be chronic.

Another symptom of depression that may be treated according to the invention is burnout.

Another symptom of depression that may be treated according to the invention is anxiety.

Another symptom of depression that may be treated according to the invention is depressive mood.

Another symptom of depression that may be treated according to the invention is withdrawal from social situations.

Another symptom of depression that may be treated according to the invention is concentration problems, which also may be denoted difficulty concentrating or poor concentration. Another symptom of depression that may be treated according to the invention is unclear, unpleasant heart palpitations.

Another symptom of depression that may be treated according to the invention is irregular heart beats.

Another symptom of depression that may be treated according to the invention is a feeling of pressure in the chest.

Another symptom of depression that may be treated according to the invention is irritable bowel syndrome, including constipation and/or diarrhea.

Another symptom of depression that may be treated according to the Invention is sexual dysfunction, such as reduced sex drive, erectile dysfunction (or male impotence) or problems with sexual desire or arousal in women.

Preferred administration forms for the iodinated activated charcoal according to the invention are tablets, tablets with disintegrants, capsules such as gelatine, vegetable or pullulan capsules, capsules which disintegrate relatively fast in the stomach such as gelatin, vegetable and pullulan capsules, wherein the tablets and capsules comprise iodine on activated charcoal and optionally comprise a bromide salt and any of flavor, colour, preservative, sweetener and excipient. In some circumstances dosage bags or a dispenser may be preferred. The iodinated activated charcoal can also be placed in a glass or cup which is filled with water or other liquid and drunk.

In some embodiments, it may be preferred to use an iodine concentration of from 1% to 20% w/w of the activated charcoal.

In some embodiments, it may be preferred to use an iodine concentration of from 2% to 15% w/w of the activated charcoal.

In some embodiments, it may be preferred to use an iodine concentration of from 3 to 10 w/w of the activated charcoal.

In some embodiments a preferred daily doses to a human of iodine administered in form of the pharmaceutical composition of the invention may be from 1 mg to 10 g.

In some embodiments a preferred daily doses to a human of iodine administered in form of the pharmaceutical composition of the invention may be from 10 mg to 1 g.

In some embodiments a preferred daily doses to a human of iodine administered in form of the pharmaceutical composition of the invention may be from 100 mg to 500 mg.

In some embodiments a preferred dose administered to a human of activated charcoal may be from 10 mg to 100 g daily.

In some embodiments a preferred dose administered to a human of activated charcoal may be from 100 mg to 10 g daily.

In some embodiments a preferred dose administered to a human of activated charcoal may be from 1 g to 5 g daily.

In some embodiments a preferred daily dose of iodine administered to a mammal in form of the pharmaceutical composition of the invention may be from 14 microgram/kg body weight to 140 mg/kg body weight.

In some embodiments a preferred daily dose of iodine administered to an mammal in form of the pharmaceutical composition of the invention may be from 140 microgram/kg body weight to 14 mg/kg body weight.

In some embodiments a preferred daily dose of iodine administered to a mammal in form of the pharmaceutical composition of the invention may be from 1.4 mg/kg body weight to 7 mg/kg body weight.

In some embodiments a preferred daily dose administered to a mammal of activated charcoal may be from 0.14 mg/kg to 1.40 g/kg body weight.

In some embodiments a preferred daily dose administered to a mammal of activated charcoal may be from 1.4 mg/kg to 0.14 g/kg body weight.

In some embodiments a preferred daily dose administered to an animal of activated charcoal may be from 14 mg/kg to 70 mg/kg body weight.

In some cases it may be preferred to co-administer a bromide salt to the mammal together with the iodinated activated charcoal. This may for example be done in order to minimize the risk of iodine toxicity.

In some embodiments it may be preferred to use one or more pharmaceutically acceptable bromide salt(s), such as sodium bromide, potassium bromide, magnesium bromide, lithium bromide, ammonium bromide and/or calcium bromide. It may be preferred that the bromide salt is impregnated into the charcoal.

In some embodiments, a preferred concentration of the co-administered bromide salt may be from 1% to 1000% w/w of the iodinated activated charcoal.

The invention will now be described in more detail by reference to some examples.

### EXAMPLES

### Example 1

### Preparation of Activated Iodinated Carbon Product of the Invention Capsules Comprising Iodine on Activated Charcoal and Sodium Bromide

Materials: Activated carbon from Sigma C7606; meets USP testing specification. Elemental Iodine from Sigma-Aldrich 03002; meets USP testing. Undenaturated ethanol From Kemetyl; meets USP and EP testing specification with <0.5% water content.

Equipment: Mixing cylinder 500 ml, measuring cylinder 500 ml, E-flask 50 ml, Buchner funnel Duran diameter 105 mm and stirrer motor with blade, RZR 1 from Heidolph. Filterpaper grade 00H from Munktell. Evaporation dish made from borosilicate glass.

Method: Depending on the batch size the amount of activated carbon, elemental iodine and ethanol is calculated. For a batch size of 50 g iodinated carbon, 4.5 g of iodine, 45.5 g of activated carbon and 450 ml ethanol is used. The activated carbon is suspended in the measuring cylinder with 410 ml ethanol and the elemental iodine is solved in the E-flask with 40 ml ethanol. The iodine is added, stirred for 2 min and allowed to impregnate the carbon for 1 h. Thereafter, the iodinated activated carbon is separated from the ethanol solution by filtration under reduced pressure and dried for 5 hrs. at 150 C. This results in iodinated activated charcoal impregnated with 9% I2.

### Example 2

### Administration of Iodine on Activated Charcoal

A male Caucasian, about 40 years old, has for some 20 years suffered from many symptoms, including chronic and severe mental fatigue in spite of 12 h sleep per night, psychological burnout, anxiety, lack of concentration, depressive mood, withdrawal from social situations, irritable bowel symptoms with constipation and/or diarrhea, anxiety, heart palpitations, pressure in the chest, reduced stamina and condition and a reduced sex drive. When asked to evaluate his subjective health status using a Quality of life questionnaire with a 1-10 scale where 1 is the worst possible and 10 is absolutely the best, he regularly marked 1 or 2. His symptoms were interpreted as caused by depression, and he was treated with 5 different types of anti-depressants for a total of 17 years. Unfortunately, none of the drugs improved his symptoms or quality of life significantly.

Some time ago, he tested the mercury binding substance iodinated activated charcoal (IAC, custom made at PharmaLundensis according to Example 1). He took 1 g per day of IAC for two weeks, and recorded the symptoms he experienced daily, as well as filling in a Quality of life questionnaire. After 2 weeks his mental fatigue had disappeared completely, he was now able to concentrate at work without effort, the anxiety, heart palpitations, pressure in the chest were better as well, and his sex drive was much improved, and he was able to function well in social situations The irritable bowel symptoms also clearly improved. His mood was considerably better and he now estimated his Quality of life score to 7-9. He felt better than he had felt for many years.

After these two weeks, he wanted to clarify if the massive improvement in his health was caused by the IAC. Therefore, he stopped taking the IAC for two weeks, which resulted in a return of the previous symptoms, and a very low Quality of life score (2-3). To clarify if it was the iodine in the IAC which caused the improvement to his health, he took elemental iodine daily in a capsule in the same amount that was present in the daily intake of the IAC (90 mg). After 2 weeks of intake of iodine capsules, his Quality of life score was as bad as when he started taking the iodine capsules, and he felt no improvements of his symptoms. To clarify if the health improvement was caused by the activated charcoal, he took, for two weeks, non-iodinated activated charcoal (0.91 g). This did not improve his health at all.

After these tests, where the two components of IAC had been tested separately and found to lack effect, the combined IAC (PharmaLundensis AB) was taken in the amount of 1 g per day. This once again improved his mood, removed the mental fatigue and symptoms of psychological burnout, the concentration problems, the anxiety, heart palpitations, pressure in the chest, irritable bowel syndrome and improved his sex drive within 2 weeks.

### Example 3

### Tablets Comprising Iodine on Activated Charcoal

Tablets comprising iodine on activated charcoal were compressed in a conventional tabletting machine from 500 mg iodinated activated charcoal mixed with 122 mg lactose monohydrate, 6 mg magnesium stearate and 122 mg sodium methyl cellulose to form a 750 mg tablet comprising about 50 mg iodine. Optionally, a pharmaceutically acceptable bromide salt, such as sodium bromide (0.5-100% w/w) can be added to the tabletting mixture,

### Example 4

### Capsules Comprising Iodine on Activated Charcoal

Capsules comprising iodine on activated charcoal were manufactured by placing 300 mg iodinated activated charcoal in capsules of size 00. The capsules can be of different type; gelatin, vegetable or pullulan, The capsules were filled with the IAC in a conventional capsule filling machine to form capsules containing about 27 mg iodine.

### Example 5

### Capsules Comprising Iodine on Activated Charcoal and Sodium Bromide

Capsules comprising iodine on activated charcoal and sodium bromide were manufactured by mixing 250 mg iodinated activated charcoal with 50 mg sodium bromide, Gelatin, vegetable or pullulan capsules were filled with the mixture in a conventional capsule filling machine to form capsules containing about 22.5 mg iodine.

1.-27. (canceled)

28. A method for treatment of one or more symptom(s) caused by depression in a mammal in need thereof, wherein said symptom(s) is/are selected from the group consisting of depressive mood, emotional fatigue, mental fatigue which can be chronic, burnout, withdrawal from social situations, anxiety, concentration problems, unpleasant heart palpitations, irregular heart beats, a feeling of pressure in the chest, irritable bowel syndrome and sexual dysfunction, the method comprising administering a pharmacologically effective amount of iodinated activated charcoal comprising elemental iodine to the intestine of said mammal.

29. The method of claim 28, wherein the iodinated activated charcoal is administered in combination with a pharmaceutically acceptable bromide salt.

30. The method of claim 29, wherein the bromide salt is sodium bromide, potassium bromide, magnesium bromide, lithium bromide, ammonium bromide and/or calcium bromide.

31. The method of claim 29, wherein the bromide salt is administered in an amount of 1-1000% w/w of the elemental iodine on the iodinated activated charcoal.

32. The method of claim 28, wherein the amount of elemental iodine administered is 1-20% w/w of the amount of the iodinated activated charcoal administered.

33. The method of claim 28, wherein the mammal is a human and wherein the human is administered a daily dose of elemental iodine from 1 mg to 10 g.

34. The method of claim 28, wherein the mammal is a human and wherein the human is administered a daily dose of iodinated activated charcoal from 10 mg to 100 g.

35. The method of claim 28, wherein the elemental iodine is administered in a daily dose of 14 μg-140 mg/kg body weight of the mammal.

36. The method of claim 28, wherein the iodinated activated charcoal is administered in a daily dose of 140 μg/kg body weight of the mammal to 1.4 g/kg body weight of the mammal.
